(19) United States
(12) Patent Application Publication
(10) Pub. No.: US 2018/0368699 A1
(43) Pub. Date: Dec. 27, 2018

(54) SYSTEM OF PREDICTING DEMENTIA AND OPERATING METHOD THEREOF

(71) Applicant: GI Signal, Ltd., Busan (KR)

(72) Inventors: Won Bae KIM, Gwacheon-si Gyeonggi-do (KR); Hunn Franz Rhee, Busan (KR); Hee Kyong YOO, Seoul (KR)

(73) Assignee: GI Signal, Ltd., Busan (KR)

(21) Appl. No.: 15/655,071

(22) Filed: Jul. 20, 2017

(30) Foreign Application Priority Data
Jun. 21, 2017 (KR) 10-2017-0078414

(51) Int. Cl. A61B 5/02; A61B 5/00; G01R 33/465; G01N 33/50

(52) U.S. Cl. CPC A61B 5/02; A61B 5/0042; A61B 5/6814; G01N 2800/2814; G01N 33/50; A61B 5/00; G01N 2333/00; G01R 33/465

(57) ABSTRACT

A dementia prediction system and an operating method thereof are disclosed. The dementia prediction system includes a bio signal collection module configured to detect brainwave information and visual information of a subject and a brain aging determination module configured to calculate directional data of a bio signal of the subject from the brainwave information and the visual information and classify the directional data into a dementia group and a normal group using training data of a preset dementia group and a preset normal group.

### CROSS-REFERENCES TO RELATED APPLICATION

The present application claims priority under 35 U.S.C. § 119(a) to Korean application number 10-2017-0078414, filed on Jun. 21, 2017, in the Korean Intellectual Property Office, which is incorporated herein by reference in its entirety.

### BACKGROUND

### 1. Technical Field

Various embodiments may generally relate to a dementia prediction system and an operating method thereof, and more particularly, to a dementia prediction system using brainwave information and visual information and an operating method thereof.

### 2. Related Art

Technology for measuring various health states of the human body using biometric signals has been studied.

Biometric signals may include, for example, electroencephalogram (EEG brainwave), electromyogram (EMG), electrocardiography (ECG), and the like. EEG among the biometric signals may refer to a waveform obtained by forming an electric field and a magnetic field through flow of current iconized by a stimulus applied to the cerebral cortex between the nerve cells and measuring minute potential change through electronics attached to the scalp. In general, EEG may be distributed in a frequency band of 0 to 100 +Hz and the potential change may be about several tens of μV and thus the amplified potential change may be recorded as EEG.

EEG may be classified into a δ-wave (4 Hz or less), a θ-wave (4-8 Hz), an α-wave (8-12 Hz), a β-wave (12-30 Hz), and a γ-wave (30-50 Hz) according to an activity state of the brain, for example, an oscillating frequency range.

EEG may also be used to diagnose a sleeping and awakened state and an abnormal state of the brain and in recent years, studies on dementia diagnosis through EEG has been actively conducted.

In recent years, with development of digital EEG measuring machines (for example, brainwave sensors), methods for diagnosing dementia through analysis and application of the brainwave have been developed. However, intervention of readers or clinical experts skilled for a reasonable period may be necessary for accurate read-out and subjective judgement criteria even between the skilled clinical experts may be different.

### SUMMARY

In an embodiment of the present disclosure, a dementia prediction system may be configured to classify a subject into a dementia group and a normal group using parameters obtained by analyzing and operating biometric information and sensory information of the subject.

In an embodiment of the present disclosure, a dementia prediction system may include a bio signal collection module configured to detect brainwave information and visual information of a subject; and a brain aging determination module configured to calculate directional data of a bio signal of the subject from the brainwave information and the visual information and classify the directional data into a dementia group and a normal group using training data of a preset dementia group and a preset normal group.

The brainwave information may include at least one of a peak frequency of an α-wave, an absolute power ratio between the α-wave and a θ-wave, and a power map of the α-wave. The visual information may include information for deposition/non-deposition of an amyloid ingredient in a retina of a human body of the subject.

In an embodiment of the present disclosure, an operating method of a dementia prediction system may include setting training data for a dementia group and a normal group by collecting biometric information and sensory information of the dementia group and collecting biometric information and sensory information of the normal group. The method may further include detecting biometric information and sensory information of a subject. The method may further include generating directional data of sensory information-reflected biometric information by combining and operating the biometric information and the sensory information of the subject. The method may further include classifying the directional data into the dementia group and the normal group using the training data. The method may further include repeatedly performing the detecting of the biometric information and the sensory information of the subject, the generating of the directional data, and the classifying of the directional data.

These and other features, aspects, and embodiments are described below in the section entitled “DETAILED DESCRIPTION”.

### BRIEF DESCRIPTION OF THE DRAWINGS

The above and other aspects, features and advantages of the subject matter of the present disclosure will be more clearly understood from the following detailed description taken in conjunction with the accompanying drawings, in which:

FIG. 1 is a block diagram illustrating a dementia prediction system according to an embodiment of the present disclosure;

FIG. 2 is a top view illustrating a brain structure of a subject;

FIG. 3 is a diagram illustrating α-wave power maps of a dementia patient and a normal person;

FIG. 4 is a graph illustrating data distributions of a dementia group and a normal group according to an embodiment of the present disclosure; and

FIGS. 5 and 6 are flowcharts illustrating an operating method of a dementia prediction system according to an embodiment of the present disclosure.

### DETAILED DESCRIPTION

Various embodiments of the present invention will be described in greater detail with reference to the accompanying drawings. The drawings are schematic illustrations of various embodiments (and intermediate structures). As such, variations from the configurations and shapes of the illustrations as a result, for example, of manufacturing techniques and/or tolerances, are to be expected. Thus, the described embodiments should not be construed as being limited to the particular configurations and shapes illustrated herein but may include deviations in configurations and shapes which do not depart from the spirit and scope of the present invention as defined in the appended claims.

The present invention is described herein with reference to cross-section and/or plan illustrations of idealized embodiments of the present invention. However, embodiments of the present invention should not be construed as limiting the inventive concept. Although a few embodiments of the present invention will be shown and described, it will be appreciated by those of ordinary skill in the art that changes may be made in these embodiments without departing from the principles and spirit of the present invention. In the drawings, the thicknesses of layers and regions may be exaggerated for clarity. In addition, like numbers refer to like element throughout.

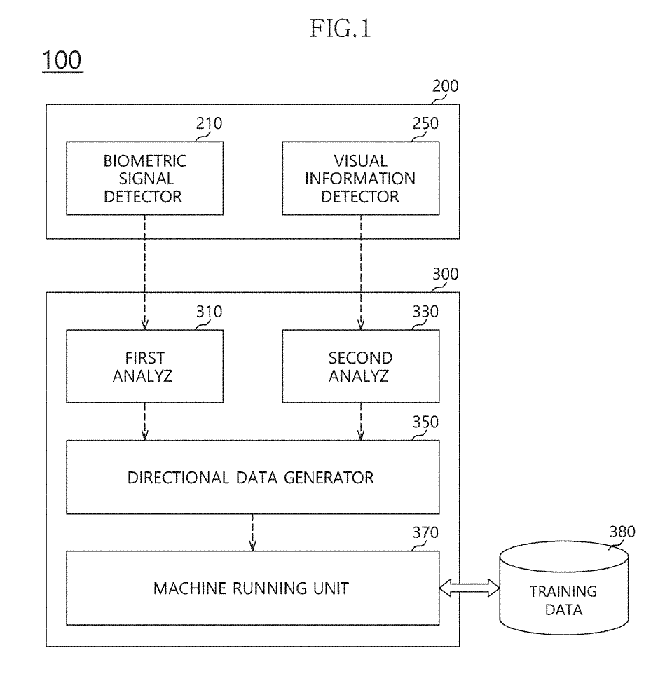

FIG. 1 is a block diagram illustrating a dementia prediction system according to an embodiment of the present disclosure.

Referring to FIG. 1, a dementia prediction system 100 according to an embodiment may include a bio signal collection module 200 and a brain aging determination module 300.

The bio signal collection module 200 may include a biometric signal detector 210 and a visual information detector 250.

The biometric signal detector 210 may include a brainwave detection sensor configured to detect a brainwave of a subject and a monitoring device. An output of the brainwave detection sensor may be, for example, a result obtained through 8 channels brainwave measuring machines for the brain.

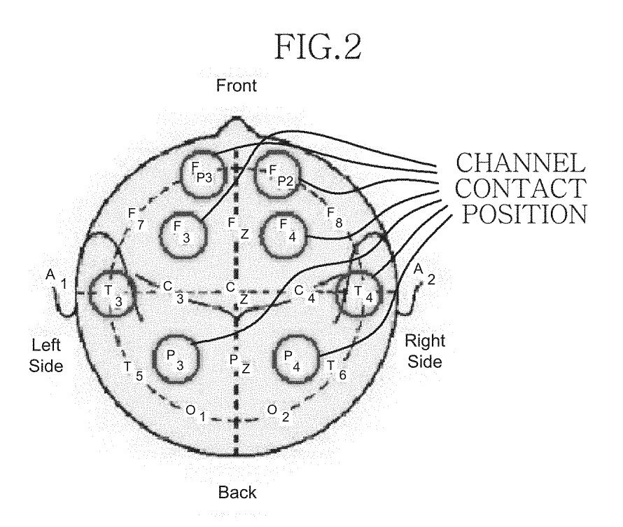

For example, when the 8 channel brainwave measuring machines are used, the brainwave detection sensor may measure the brainwave of the subject using brainwave detection channels (not shown) attached to a brain structure of the subject, for example, 8 portions selected from frontal poles Fp1 and Fp2, frontal portions F3 and F4, central portions C3 and C4, parietal portions P3 and P4, occipital portions O1 and O2, anterior temporal portions F7 and F8, middle temporal portions T3 and T4, posterior temporal portions T5 and T6, a midline-frontal portion Fz, a midline-central portion Cz, and a midline-parietal Pz as illustrated in FIG. 2. A δ-wave, a θ-wave, an α-wave, a β-wave, a γ-wave, and the like may be extracted in each channel. Frequencies having all the extracted wavelengths may be used for supervised learning of dementia prediction diagnosis, but the α-wave and the θ-wave may be mainly used as important parameters for aging state analysis in the embodiment. An output of the biometric signal detector 210 may be, for example, brainwave information detected in a steady state.

For example, the visual information detector 250 may include an eye imaging device. In another example, the visual information detector 250 may include an eye image device and a monitoring device configured to monitor various pieces of visual information detected through the eye imaging device. The visual information detector 250 may detect a check signal of β-amyloid deposited in the retina through the eye image device. The β-amyloid check signal may be a biomarker for sensing a brain aging function.

In general, the deposition of the β-amyloid material in the brain may be a distinct symptom of dementia. However, the deposition of the β-amyloid material in the brain may be founded when the dementia progresses to a certain degree. Since the method for detecting the deposition of the β-amyloid material in the brain is very complicated and is painful to the subject, the early diagnosis of dementia may be difficult.

Unlike other structures of the eye, the retina of the human body is a part of the central nervous system and is known to share many features of the brain. Accordingly, when the retina of the subject is shoot through the eye imaging device, the deposition of the β-amyloid material in the retina may be easily detected. Since the β-amyloid material is earlier deposited in the retina than in the brain, the abnormality of the brain may be early detected. Accordingly, the deposition of the β-amyloid material in the retina may be used as a parameter for determining aging of the brain and thus the test may be easily conducted without pain of the subject unlike a brain test for example, a lumbar puncture or a magnetic resonance imaging (MRI) test.

The eye imaging device may shoot the retina using the hyperspectral imaging technique. The hyperspectral imaging method may be a method for radiating light to the eyeball of the human body, reflecting the light reaching the retina behind the eyeball, and displaying a retinal image using a wavelength of the reflected light.

The deposition/non-deposition of the β-amyloid material in the retina is used as the visual information of the human body in the embodiment, but this is not limited thereto. A variation in widths of the retinal vessels measured through an image sensor of the eye imaging device may be used as the visual information. The widths of the retinal vessels may be values based on the artery and vein of the retina.

The brain aging determination module 300 may include a first analyzer 310, a second analyzer 330, a directional data generator 350, and a machine running unit 370.

The first analyzer 310 may generate parameters necessary for analysis by receiving the brainwave information detected from the biometric signal detector 210.

The first analyzer 310 may generate a waveform feature of the α-wave from various pieces of information input from the biometric signal detector 210. For example, the first analyzer 310 may generate parameters, for example, frequency where the power is maximal within the alpha spectrum (hereinafter, referred to as “peak frequency information of the α-wave”), an absolute power ratio P_{α/θ} between the α-wave and the θ-wave, a power map of the α-wave, an average, dispersion, kurtosis, and skewness of the α-wave spectrum, and the like.

First, the peak frequency information of the α-wave may be a very important factor in determining a function of the brain. For example, when the brain is aged or is closer to dementia, the peak frequency information of the α-wave may be represented to be slow. When the brain function is good, the peak frequency information of the α-wave may be represented to be high in a range of 8 to 12 Hz which is the frequency range of the α-wave. Since the α-wave is measured in the two portions of the frontal lobe in the steady state as the intrinsic rhythm, signals measured through channels which are in contact with the two portions of the frontal lobe (for example, two portions Fp1 and Fp2 of frontal pole in FIG. 2) may be used.

It is known that as the aging function of the brain is lowered, the θ-wave power increases in the steady state thus the ratio of θ-wavepower to α-wave power decreases. This is because the cognitive function is injured and thus the brainwave of a low frequency band is relatively activated. Accordingly, the absolute power ratio P_{α/θ} between the α-wave and the θ-wave in the following Equation 1 may be an another measure in determining the aging of the brain.

P_{α/θ}=(P_{α}^{Fp1}+P_{α}^{Fp2})/(P_{θ}^{FP1}+P_{θ}^{FP2}) [Equation 1]

Here, P_{α}^{Fp1}: absolute power of the α-wave in a first frontal pole,

P_{α}^{Fp2}: absolute power of the α-wave in a second frontal pole,

P_{θ}^{FP1}: absolute power of the θ-wave in the first frontal pole, and

P_{θ}^{FP2}: absolute power of the θ-wave in the second frontal pole.

The absolute power corresponds to the integral of the power spectrum.

The power map of the α-wave may be calculated by an absolute power value of the α-wave obtained through the 8 channels of the biometric signal detector 210. For example, since the absolute power value of the α-wave of a person having a degraded brain function or a dementia patient which is obtained through each channel is reduced as compared with a normal person, such variation amount may be used as a dementia prediction parameter.

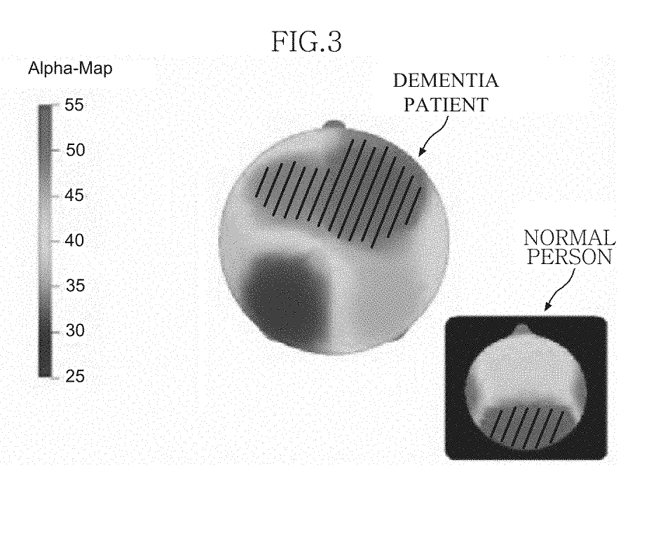

As illustrated in FIG. 3, the spatial distribution of the α-wave power in the dementia patient may be represented to be different from the normal person. For example, it can be seen that the α-wave power of the severe dementia patient is biasedly distributed in the frontal lobe portion rather than the occipital lobe portion and thus the power map of the α-wave may be used as the dementia prediction parameter.

Further, the average and dispersion of the α-wave power spectrum may be used as the dementia prediction parameter. Here, the average of the α-wave power spectrum refers to the average frequency of the power spectrum of the α-wave and the dispersion of the α-wave refers to the extent of spread (for example, a width) of the α-wave power spectrum. For example, as the dementia progresses, average frequency of the α-wave power spectrum may be lowered and the dispersion of the α-wave power spectrum may be widely distributed.

The second analyzer 330 may receive data indicating whether or not the β-amyloid material is deposited in the retina from the eye imaging device of the visual information detector 250 and output a signal of logic 1 in deposition of the β-amyloid material and a signal of logic 0 in non-deposition of the β-amyloid material by processing the received data for the deposition of the β-amyloid material.

The directional data generator 350 may receive the parameter calculated in the first analyzer 310 and the parameter calculated in the second analyzer 330 and form a feature vector for each parameter and a vector space through a combination of the parameters.

For example, the directional data generator 350 may form a feature vector \vec{f} for dementia prediction and may have following features which are depicted in Equation 2.

\vec{f}=(\hat{α}, P_{α/θ}, \vec{P_{α}}, aᵥₐᵣ, R_{β}) [Equation 2]

Here, \hat{α}: peak frequency of the α-wave,

P_{α/θ}: absolute power ratio between the α-wave and the θ-wave,

\vec{P_{α}}: power map of the α-wave,

αₘₑₐₙ: average frequency of the α-wave power spectrum,

αaᵥₐᵣ: dispersion of the α-wave power spectrum, and

R_{β}: pigmentation/non-pigmentation of the β-amyloid in the retina.

The directional data generator 350 may induce the feature space (F, F [symbol] \vec{f}) for dementia prediction from the following Equation 3.

F=I_{\hat{α}}×I_{pα/θ}×I_{β}_{_{α}}×I_{α}_{ₘₑₐₙ}×I_{α}_{ᵥₐᵣ}×I_{R}_{_{β}} [Equation 3]

Here, I indicates a variable range (distribution) of each parameter and may refer to an effective interval range (effective distribution) for each parameter.

For example, the peak frequency of the α-wave may be corresponding to 8 to 12 Hz and thus the peak frequency distribution I_{\hat{α}} of the α-wave may be set [8, 12]. The distribution of the absolute power ratio between the α-wave and the θ-wave may be set to [0.1, 3] by considering that the absolute power ratio between the α-wave and the θ-wave in the normal person is 1.5 or more. For example, since the dementia patient has a low peak level of the α-wave and has a high appearance ratio of the θ-wave, the absolute power ratio of the dementia patient may be set between 0.1 and 3. The power map distribution I_{\hat{P}}_{_{α}}of the α-wave may be set to [0, 100] for the 8 channels and the average distribution I_{α}_{ₘₑₐₙ} of the α-wave may be set to [8, 12] which is the frequency range of the α-wave. The dispersion distribution I_{α}_{ᵥₐᵣ} of the α-wave may be set to [0, 4²] through the dispersion equation by considering that the dispersion distribution I_{α}_{ᵥₐᵣ} of the α-wave in the normal person is between 0 and 4. The deposition/non-deposition I_{R}_{_{β}} of the β-amyloid in the retina may be set to [0, 1].

The feature vector of the individual subject may be formed of a space having an effective interval for each parameter, that is, a feature space. The feature space by Equation 3 may be represented with a high-dimensional space, for example, a 13-dimensional space.

The directional data generator 350 may extract the directional data of the biometric information to which the visual information of the subject is reflected by analyzing and operating the biometric signal information and the visual information detected from the subject.

The machine running unit 370 may group similar data entities by receiving the directional data of the biometric information to which the visual information for each subject is reflected. The machine running unit 370 in the embodiment may use supervised learning.

The supervised learning may be a task which derives one function from the training data and may be a series of processes for determining whether the directional data of the subject has a more similar type to which group of data using the training data which is classified into the directional data of the dementia group and the directional data of the normal group on a data base 380.

The training data may be a vector indicating the directivity of the biometric information to which the visual information obtained under the same condition as the directional data measured from the subject is reflected. The training data may be represented to be included in which group through the supervised learning. The term “classification” may refer to a task for representing that the vector of an input entity of the derived function is which type of value. The dementia may be predicted based on the supervised leaning according to the concept of the present disclosure.

The machine running unit 370 in the embodiment may classify the high-dimensional feature space into the normal group and the dementia group and the classification into the normal group and the dementia group will be defined as the following Equation 4.

C={C1,C2} [Equation 4]

Here, C1 refers to the normal group and C2 refers to the dementia group.

When the machine running unit 370 provides (\vec{f},C) by pairing the normal group of the training data to C1 and pairing the dementia group of the training data to C2, the accurate prediction result may be obtained according to the classified result through a support vector machine (SVM) algorithm.

The SVM algorithm may be typically trained through the supervised learning and may be calculated as the best function which associates the output result with the input data. The SVM algorithm is described in detail in Bernhard Schoelkopf, Alex Smola, “Learning with Kernels” MIT Press, Cambridge, Mass. 2002.

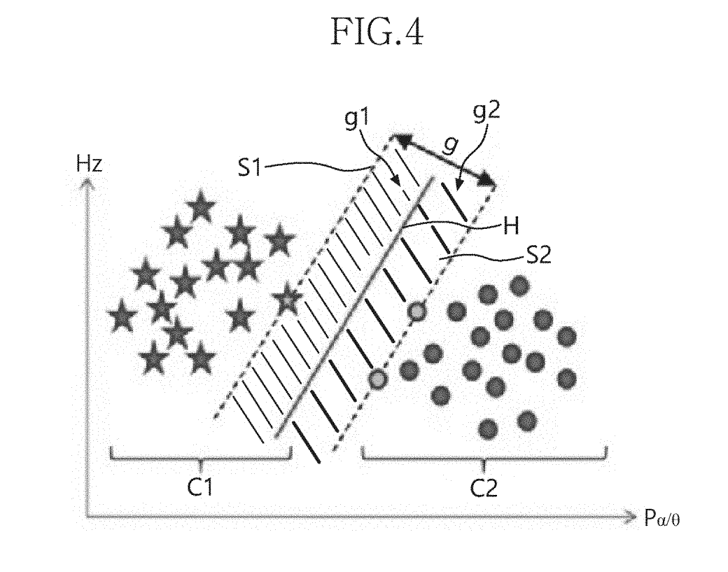

Referring to FIG. 4, the input data may be classified into the normal group and the dementia group in the classes C1 and C2 with the minimal error through the SVM algorithm.

The classified group may be formed based on the training data through the machine running process and when target data of an actual subject is input, the target data may be classified to be is included in which group.

For the enhancement and refinement of an analysis signal, it is important to set a gap g between a hyperplane H and support vectors S1 and S2. The hyperplane H may be corresponding to an intermediate value of the gap g between the class C1 and the class C2, the support vector S1 may indicate a boundary line of the class C1 which is closest to the hyperplane H, and the support vector S2 may indicate a boundary line of the class C2 which is closest to the hyperplane H.

As described above, the hyperplane H may be typically set to the intermediate value of the gap g, but the setting of the hyperplane H may be changed by checking the similarity and the degree of correlation within the classified group.

As the number of times that the training data is cumulated is increased, a quality of basic data is increased and thus the machine running unit 370 may accomplish accurate dementia prediction. For example, pieces of directional data of the plurality of subjects may be included in the training data and thus boundary lines of distributions of the classes 1 and 2 C1 and C2 may be more cleared and the probability of error may be further reduced.

For the enhancement and refinement of an analysis signal in the feature vector calculation process, the kurtosis and skewness of the α-wave other than the average I_{α}_{ₘₑₐₙ} and dispersion I_{α}_{ᵥₐᵣ} of the α-wave power spectrum may be selected as the feature. The kurtosis and skewness may be an important factor for characterizing the dementia phenomenon.

For example, when the waveform of the α-wave has no Gaussian form and is concentrated on an one point or when the waveform of the α-wave has a Gaussian form and the center axis thereof is biased to one side, the kurtosis αₖᵤᵣₜₒₛᵢₛ and skewness α_{skew} of the α-wave power spectrum may be reflected additionally in the feature vector and the vector component of the feature space as in the following Equation 5. The kurtosis and skewness of the α-wave power spectrum may be obtained in the first analyzer 310 through the detection result of the biometric signal detector 210.

\vec{f}=(\hat{α}, P_{α/θ}, \vec{P_{α}}, αₖᵤᵣₜₒₛᵢₛ, α_{skew}, R_{β})

F=I_{\hat{α}}×I_{pα/θ}×I_{\vec{P}}_{_{α}}×I_{α}_{ₖᵤᵣₜₒₛᵢₛ}×I_{α}_{_{skew}}×I_{R}_{_{β}} [Equation 5]

The deposition/non-deposition of β-amyloid in the retina is used as the visual information in the embodiment, but a parameter through a pupil light reflection test may be used as the visual information.

Several parameters may be obtained through pupilometry using image analysis technology in a pupillary light reflex (PLR) test such as a standby time to pupil reflection. Accordingly, the subjects may be mapped using feature vectors of Equations 6 and 7.

\vec{f}=(\hat{α}, P_{α/θ}, \vec{P_{α}}, αₖᵤᵣₜₒₛᵢₛ, α_{skew}, E_{PLR}) [Equation6]

Here, EPLR indicates a variable obtained from the PLR test.

\vec{f}=(\hat{α}P_{α/θ}, \vec{P_{α}}, αₘₑₐₙ, αᵥₐᵣ, αₖᵤᵣₜₒₛᵢₛ, α_{skew}, R_{β}, E_{PLR}) [Equation 7]

The feature parameters may be added or omitted according to best input for the machine learning algorithm.

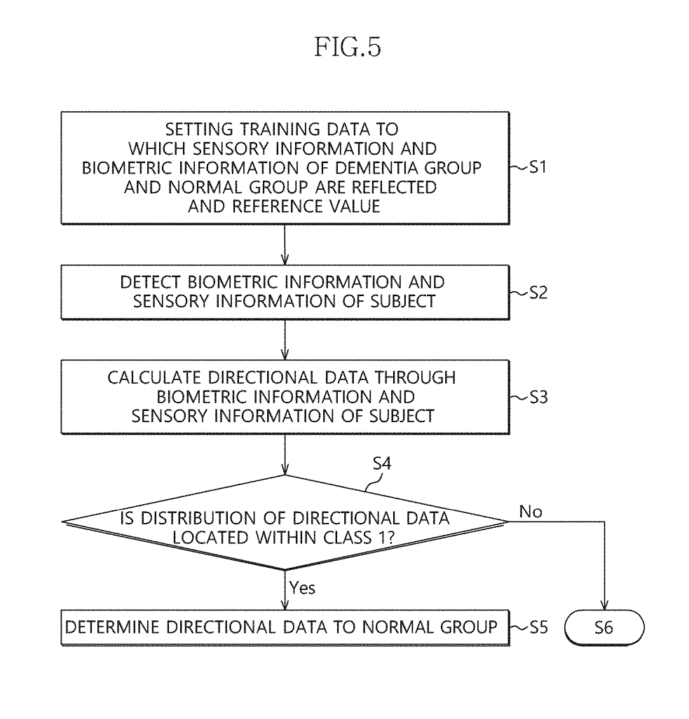

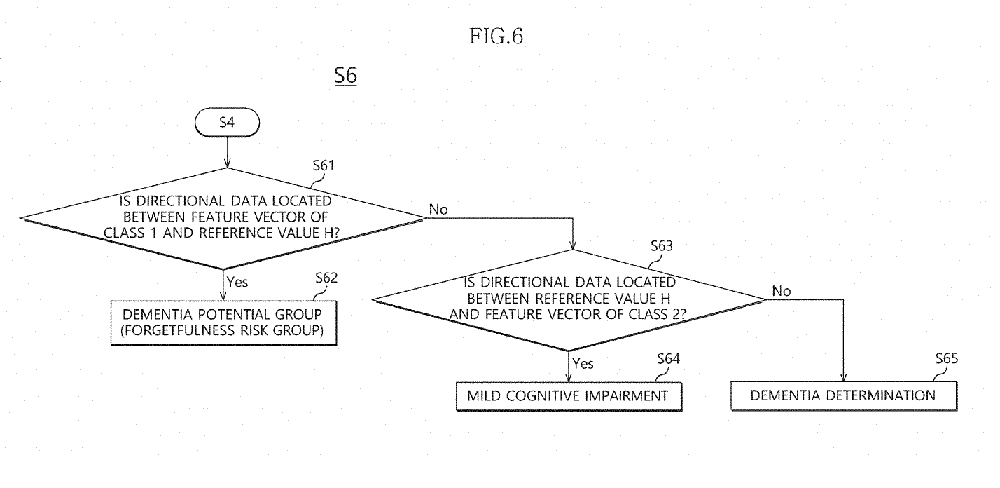

An operating method of a dementia prediction system according to an embodiment will be described with reference to FIGS. 5 and 6.

Referring to FIG. 5, first, the dementia prediction system may set training data for a dementia group and a normal group by collecting biometric information and sensory information of a dementia group and collecting biometric information and sensory information of a normal group (S1). The training data may be stored in a database form. Here, as described above, the biometric information may be the brainwave information and the sensory information may be the visual information.

The dementia prediction system may detect biometric information and sensory information of a subject under the same condition as the training data (S2).

The dementia prediction system may extract main parameters of the biometric information and the sensory information and generate directional data of the sensory information-reflected biometric information through combination and operation of the main parameters in the brain aging determination module 300 (S3).

The dementia prediction system may classify the directional data into a class 1 (normal group) or a class 2 (dementia group) based on the training data on the database (S4, S5, and S6).

The classification process will be described below in detail. First, the dementia prediction system may determine whether or not the directional data is located within the class 1 C1 (S4). When the directional data of the subject is located within the class 1 C1, the machine running unit 370 may determine the subject to the normal group (S5). For example, the feature vector may be one entity displayed in the feature space and the support vector may correspond to a portion of the feature vectors which determine the hyperplane that separates the two classes.

When the directional data of the subject is not located within the class 1 C10 (S6), as illustrated in FIGS. 4 and 6, the machine running unit 370 may determine whether or not the directional data is located between the feature vector of the class 1 C1 and the hyperplane H (S61). When the directional data of the subject is located between a gap g1 between the feature vector of the class 1 C1 and the hyperplane H, the subject may be not a dementia risk group, but the subject may have a dementia potential factor as compared with the normal person. Accordingly, the machine running unit may determine the subject to a dementia potential group or a forgetfulness risk group (S62).

When the directional data of the subject is locate in a gap g2 between the hyperplane H and the class 2 C2 (S63), the machine running unit may determine the subject not to a complete dementia state but to the dementia risk group such as mild cognitive impairment (S64). When the directional data of the subject is not located between the hyperplane H and the feature vector of the class 2 C2 and is corresponding to the class 2 C2, the machine running unit may determine the subject to dementia.

As described above, the error range may be reduced by repeatedly performing operations S2 to S6 and thus more accurate training data may be ensured. For example, the classification-completed directional data of the subject may be continuously reflected to the training data for a reference and thus the more accurate training data for the reference may be formed.

The brainwave detection sensor is used as the bio signal collection module 200 in the embodiment, but this is not limited thereto. The human body signal may be collected through a heartrate variability (HRV) sensing and electromyogram sensor.

According to an embodiment, the dementia group and the normal group may be more accurately classified using the feature vector, which is formed through a combination of the brainwave information as biometric information and the visual information, as the training data.

The subject may be classified into mild cognitive impairment, a dementia potential group (forgetfulness risk group), and a normal group through the feature vector and the feature space obtained from the feature vector, for example, a distance from the hyperplane H and distributions.

The directional data of the subject may be updated as the training data and thus the clearer data reference value may be provided.

The above described embodiments of the present invention are intended to illustrate and not to limit the present invention. Various alternatives and equivalents are possible. The invention is not limited by the embodiments described herein. Nor is the invention limited to any specific type of semiconductor device. Other additions, subtractions, or modifications are obvious in view of the present disclosure and are intended to fall within the scope of the appended claims.

What is claimed is:

1. A dementia prediction system comprising:
a module configured to classify a subject into a dementia group and a normal group using parameters obtained by analyzing and operating biometric information and sensory information of the subject.

2. The dementia prediction system of claim 1, wherein the module includes:
a bio signal collection module configured to detect the biometric information and the sensory information; and
a brain aging determination module configured to generate directional data for determining whether or not a brain of the subject is aged based on a detection result of the bio signal collection module.

3. The dementia prediction system of claim 2, wherein the bio signal collection module includes:
a biometric signal detector configured to detect brainwave information of a human body of the subject; and
a visual information detector configured to detect visual information of the human body.

4. The dementia prediction system of claim 3, wherein the visual information detector is configured to detect at least one of deposition/non-deposition of a β-amyloid material in a retina of the human body, a width of a retinal vessel, and a pupillary light reflex (PLR) test result.

5. The dementia prediction system of claim 2, wherein the brain aging determination module includes:
a first analyzer configured to process data detected from the biometric signal detector;
a second analyzer configured to process data detected from the visual information detector;
a directional data generator configured to generate the bidirectional data by combining and operating parameters extracted from the first and second analyzers; and
a machine learning unit configured to classify the directional data into the dementia group and the normal group by grouping similar data entities.

6. The dementia prediction system of claim 5, wherein the first analyzer is configured to generate at least one among a peak frequency parameter of an α-wave of the brain, an absolute power ratio parameter between the α-wave and a θ-wave of the brain, and a power map parameter of the α-wave of the brain through a detection result of the biometric signal detector.

7. The dementia prediction system of claim 5, wherein the first analyzer is configured to calculate an average and dispersion of an α-wave power spectrum of the brain through a detection result of the biometric signal detector and provide the average and dispersion of the α-wave power spectrum of the brain to the directional data generator.

8. The dementia prediction system of claim 5, wherein the first analyzer is configured to calculate kurtosis and skewness distributions of an α-wave power spectrum of the brain through a detection result of the biometric signal detector and provide the kurtosis and skewness distributions of the α-wave power spectrum of the brain to the directional data generator.

9. The dementia prediction system of claim 5, wherein the second analyzer is configured to perform signaling on deposition/non-deposition of a β-amyloid ingredient in a retina of the subject through a detection result of the visual information detector and provide a signaling result to the directional data generator.

10. The dementia prediction system of claim 5, wherein the directional data generator is configured to generate a feature vector and a feature vector space by combining the parameters provided from the first and second analyzers.

11. The dementia prediction system of claim 5, wherein the machine learning unit is configured to classify the directional data of the subject using training data directivity-databased through detection of the biometric signals and visual information of a dementia patient and a normal person and reflect a classified result to the training data.

12. A dementia prediction system comprising:
a bio signal collection module configured to detect brainwave information and visual information of a subject; and
a brain aging determination module configured to calculate directional data of a bio signal of the subject from the brainwave information and the visual information and classify the directional data into a dementia group and a normal group using training data of a preset dementia group and a preset normal group.

13. The dementia prediction system of claim 12, wherein the brainwave information includes at least one of a peak frequency of an α-wave, an absolute power ratio between the α-wave and a θ-wave, and a power map of the α-wave.

14. The dementia prediction system of claim 12, wherein the visual information includes information for deposition/non-deposition of a β-amyloid ingredient in a retina of a human body of the subject.

15. A method of operating a dementia prediction system, the method comprising:
setting training data for a dementia group and a normal group by collecting biometric information and sensory information of the dementia group and collecting biometric information and sensory information of the normal group;
detecting biometric information and sensory information of a subject;
generating directional data of sensory information-reflected biometric information by combining and operating the biometric information and the sensory information of the subject;
classifying the directional data into the dementia group and the normal group using the training data; and
repeatedly performing the detecting of the biometric information and the sensory information of the subject, the generating of the directional data, and the classifying of the directional data.

16. The method of claim 15, wherein the classifying of the directional data using the training data includes:
determining the directional data as normal when the directional data of the subject is located in a range of the normal group; and
determining the directional data as dementia when the directional data of the subject is located in a range of the dementia group.

17. The method of claim 16, wherein the classifying of the directional data using the training data further includes:
setting an intermediate value between the range of the dementia group and the range of the normal group as a reference value;
determining a subject having directional data located between the reference value and the range of the normal group to a dementia potential group; and
is determining a subject having directional data located between the reference value and the range of the dementia group to a dementia risk group.

18. The method of claim 15, wherein the generating of the directional data of the sensory information-reflected biometric information by combining and operating the biometric information and the sensory information of the subject includes forming a feature vector and a feature vector space using the biometric information and the sensory information.

19. The method of claim 15, wherein the directional data of the subject is reflected to the training data through the repeatedly performing.
